(19) United States
(12) Patent Application Publication
(10) Pub. No.: US 2003/0162709 A1
(43) Pub. Date: Aug. 28, 2003

(54) Methods of generating multispecific, multivalent agents from VH and VL domains

(76) Inventors: Edmund Rossi, Morris Plains, NJ (US); Chien-Hsing Ken Chang, Morris Plains, NJ (US); David Goldenberg, Mendham, NJ (US)

Correspondence Address: FOLEY AND LARDNER, SUITE 500, 3000 K STREET NW, WASHINGTON, DC, 20007, US

(21) Appl. No.: 10/328,190

(22) Filed: Dec. 26, 2002

Related U.S. Application Data
(60) Provisional application No. 60/342,103, filed on Dec. 26, 2001.

(51) Int. Cl. A61K 39/395; C07K 16/30

(52) U.S. Cl. 514/12; 424/155.1; 530/388.8

(57) ABSTRACT

This invention relates to multi-specific, multivalent binding proteins and methods of generating these agents from V_{H} and V_{L} domains. The binding protein has three or more binding sites where at least one binding site binds with a hapten moiety and at least two sites bind with target antigens. The present invention further relates to bispecific, trivalent heterodimers that have at least one binding site with affinity towards molecules containing a histamine-succinyl-glycyl (HSG) moiety and at least two binding sites with affinity towards carcinoembryonic antigen (CEA), and to trispecific, trivalent heterodimers that have at least one binding site with affinity towards molecules containing a HSG moiety, at least one binding sites with affinity towards CEA, and at least one binding site having affinity towards a metal-chelate complex indium-DTPA. Moreover, this invention relates to recombinant vectors useful for the expression of these functional heterodimers in a suitable host.

### FIELD OF THE INVENTION

This invention relates to multi-specific, multivalent binding proteins and methods of generating these agents from V_{H} and V_{L} domains. The binding protein has three or more binding sites where at least one binding site binds with a hapten moiety and at least two sites bind with target antigens. The present invention further relates to bispecific, trivalent heterodimers that have at least one binding site with affinity towards molecules containing a histamine-succinyl-glycyl (HSG) moiety and at least two binding sites with affinity towards carcinoembryonic antigen (CEA), and to trispecific, trivalent heterodimers that have at least one binding site with affinity towards molecules containing a HSG moiety, at least one binding sites with affinity towards CEA, and at least one binding site having affinity towards a metal-chelate complex indium-DTPA. Moreover, this invention relates to recombinant vectors useful for the expression of these functional recombinant proteins in a host cell.

### BACKGROUND OF THE INVENTION

Man-made binding proteins, in particular monoclonal antibodies and engineered antibodies or antibody fragments, have been tested widely and shown to be of value in detection and treatment of various human disorders, including cancers, autoimmune diseases, infectious diseases, inflammatory diseases, and cardiovascular diseases [Filpula and McGuire, Exp. Opin. Ther. Patents (1999) 9: 231-245]. For example, antibodies labeled with radioactive isotopes have been tested to visualize tumors after injection to a patient using detectors available in the art. The clinical utility of an antibody or an antibody-derived agent is primarily dependent on its ability to bind to a specific targeted antigen. Selectivity is valuable for delivering a diagnostic or therapeutic agent, such as isotopes, drugs, toxins, cytokines, hormones, growth factors, conjugates, radionuclides, or metals, to a target location during the detection and treatment phases of a human disorder, particularly if the diagnostic or therapeutic agent is toxic to normal tissue in the body.

The major limitations of antibody systems are discussed in Goldenberg, The American Journal of Medicine (1993) 94: 298-299. The preferred parameters in the detection and treatment techniques are the amount of the injected dose specifically localized at the site(s) where target cells are present and the uptake ratio, i.e. the ratio of the concentration of specifically bound antibody to that of the radioactivity present in surrounding normal tissues. When an antibody is injected into the blood stream, it passes through a number of compartments as it is metabolized and excreted. The antibody must be able to locate and bind to the target cell antigen while passing through the rest of the body. Factors that control antigen targeting include location, size, antigen density, antigen accessibility, cellular composition of pathologic tissue, and the pharmacokinetics of the targeting antibodies. Other factors that specifically affect tumor targeting by antibodies include expression of the target antigens, both in tumor and other tissues, and bone marrow toxicity resulting from the slow blood-clearance of the radiolabeled antibodies.

The amount of targeting antibodies accreted by the targeted tumor cells is influenced by the vascularization and barriers to antibody penetration of tumors, as well as intratumoral pressure. Non-specific uptake by non-target organs such as the liver, kidneys or bone-marrow is another potential limitation of the technique, especially for radioimmunotherapy, where irradiation of the bone marrow often causes the dose-limiting toxicity.

One suggested solution, referred to as the “Affinity Enhancement System” (AES), is a technique especially designed to overcome the deficiencies of tumor targeting by antibodies carrying diagnostic or therapeutic radioisotopes [U.S. Pat. No. 5,256,395 (1993), Barbet et al., Cancer Biotherapy & Radiopharmaceuticals (1999) 14: 153-166]. The AES requires a radiolabeled bivalent hapten and an anti-tumor/anti-hapten bispecific antibody that recognizes both the target tumor and the radioactive hapten. The technique involves injecting the bispecific antibody into the patient and allowing the bispecific antibody to localize at the target tumor. After a sufficient amount of time for the unbound antibody to clear from the blood stream, the radiolabeled hapten is administered. The hapten binds to the antibody-antigen complex located at the site of the target cell to obtain diagnostic or therapeutic benefits. The unbound hapten clears the body. Barbet mentions the possibility that a bivalent hapten may crosslink with a bispecific antibody, when the latter is bound to the tumor surface. As a result, the radiolabeled complex is more stable and stays at the tumor for a longer period of time.

Additionally, current methods for generating bispecific or trispecific triabodies pose problems. These methods teach the synthesis of three distinct polypeptides, each consisting of a V_{H} domain directly linked to a V_{L} domain. For a bispecific triabody that is bivalent for the specificity of V_{H1}/V_{L1} and monovalent for the specificity of V_{H2}/V_{L2}, the three polypeptides would be V_{H1}-V_{L2}, V_{H2}-V_{L1}, and V_{H1}-V_{L1}. For a trispecific triabody that is monovalent for each of the three specificities (V_{H1}/V_{L1}, V_{H2}/V_{L2}, and V_{H3}/V_{L3}), the three polypeptides would be V_{H1}-V_{L2}, V_{H2}-V_{L3}, and V_{H3}-V_{L1}. Since each polypeptide of either design has the potential of forming a triabody by associating with itself or with the two other polypeptides, up to 10 distinct triabodies may be produced, with only one being the correct structure. Similar approaches to producing a multispecific tetramers based on the tetrabody concept would only magnify the number of potential side-products by adding a fourth polypeptide.

Moreover, multispecific, multivalent designs, such as the tandem diabody, also suffers a potential drawback. It is not unlikely that with other antibodies of choice, a homodimer may not form readily if the polypeptide consisting of both V_{H} and V_{L} domains of two different antibodies can fold back onto itself to yield a bispecific single chain with monovalency for each specificity. In fact, a few constructs have been made based on the tandem diabody design that produced a bispecific single chain structure, instead of a tandem diabody, in each case (Rossi and Chang, unpublished results). Therefore, intra-chain pairing of V_{H} and V_{L} domains is a distinct possibility when both types are present on the same polypeptide, especially when the distance between the cognate V_{H} and V_{L} domains is sufficiently long and flexible.

Bispecific, multivalent antibodies prepared by chemically crosslinking two different Fab′ fragments have been employed successfully, along with applicable bivalent haptens, to validate the utility of the AES for improved tumor targeting both in animal models and in human patients. However, there remains a need in the art for production of bispecific antibodies by recombinant DNA technology that are useful in an AES. Specifically, there remains a need for an antibody that exhibits enhanced antibody uptake at targeted antigens, decreased antibody in the blood, optimal protection of normal tissues and cells from toxic pharmaceuticals. Moreover, there remains a need for binding proteins that overcome the problems associated with generating scFv-based agents of multivalency and multispecificity.

### SUMMARY OF THE INVENTION

This invention relates to multi-specific, multivalent binding proteins and methods of generating these agents from V_{H} and V_{L} domains. The binding protein has three or more binding sites where at least one binding site binds with a hapten moiety and at least two sites bind with target antigens. The present invention further relates to bispecific, trivalent proteins that have at least one binding site with affinity towards molecules containing a histamine-succinyl-glycyl (HSG) moiety and at least two binding sites with affinity towards carcinoembryonic antigen (CEA), and to trispecific, trivalent binding proteins that have at least one binding site with affinity towards molecules containing a HSG moiety, at least one binding sites with affinity towards CEA, and at least one binding site having affinity towards a metal-chelate complex indium-DTPA. Moreover, this invention relates to recombinant vectors useful for the expression of these functional binding proteins in a host (preferably a microbial host).

One embodiment of the present invention relates to bispecific, trivalent heterodimers that bind with hapten moieties and target antigens and to recombinant vectors useful for the expression of these functional recombinant proteins in a host (preferably microbial host).

A second embodiment is a bispecific, trivalent heterodimer that has at least one binding site with affinity towards molecules containing a HSG moiety and at least two binding sites with affinity towards CEA, and to recombinant vectors useful for the expression of these functional heterodimers in a host (preferably a microbial host). These heterodimers are produced via recombinant DNA technology and create a novel AES that shows specific affinity for HSG and CEA.

A third embodiment is a trispecific, trivalent heterodimer that has at least one binding site with affinity towards molecules containing a HSG moiety, at least one binding site with affinity towards CEA, and at least one binding site having affinity towards a metal-chelate complex indium-DTPA. This embodiment includes to recombinant vectors useful for the expression of these functional heterodimers in a microbial host. These heterodimers are produced via recombinant DNA technology and create a novel AES.

A fourth embodiment of this invention relates to a method of delivering a diagnostic agent, a therapeutic agent, or a combination thereof to a target. The method includes administering to a subject in need of the agent with the binding protein, waiting a sufficient amount of time for an amount of the non-binding protein to clear the subject's blood stream, and administering the carrier molecule. A further embodiment of the present invention is a method of detecting or treating a human disorder with the method of delivering the agent to a target.

It is an object of the present invention to produce a binding protein that is capable of binding with hapten moieties and antigens. It is yet a further object of this invention to produce vectors that contain sequences of DNA encoding for multi-specific antibodies and that are readily expressed in microbial host cells. Moreover, this invention includes a method of producing a heterodimer by recombinant DNA technology. The method includes culturing the host cell in a suitable media and separating the heterodimer from the media. Further, the invention relates to a nucleic acid molecule selected from the group of cDNA clones consisting of a polynucleotide encoding the polypeptides contained in FIGS. 4-7 (Seq IDs). The DNA coding sequence of nucleic acids and the corresponding encoded amino acids for 679-scFv-L5 and hMN14-scFv-L5 are contained in FIGS. 4 and 6 (Seq IDs), respectively. The DNA coding for m734 V_{H} and V_{L} are in FIG. 7.

### BRIEF DESCRIPTION OF THE DRAWINGS

FIG. 1 shows a schematic representation of the 679 single chain Fv (scFv) polypeptide that is synthesized in E. coli from the 679-scFv-L5 expression plasmid and forms a 679 heterodimer. The gene construct for the un-processed polypeptide contains the pelB signal peptide, 679V_{H} and V_{K} coding sequences coupled by a 5 amino acid linker, Gly-Gly-Gly-Gly-Ser (G₄S), and the carboxyl terminal six histidine (His) affinity tag. The figure also shows a stick figure drawing of the mature polypeptide after proteolytic removal of the pelB leader peptide and a stick figure drawing of a 679 heterodimer, including the HSG binding sites.

FIG. 2 shows a schematic representation of the hMN14scFv polypeptide that is synthesized in E. coli from the hMN14-scFv-L5 expression plasmid and forms a hMN14 heterodimer. The gene construct for the un-processed polypeptide contains the pelB signal peptide, hMN14V_{H} and V_{K} coding sequences coupled by a 5 amino acid linker, and the carboxyl terminal 6 histidine affinity tag. The figure also shows a stick figure drawing of the mature polypeptide following proteolytic removal of the pelB leader peptide, and a stick figure drawing of a hMN14 heterodimer, including CEA binding sites.

FIG. 3 shows a schematic representation of the m734-scFv polypeptide that is to be synthesized in E. coli from the 734-scFv-L5 expression plasmid and can form a 734 heterodimer. The gene construct for the un-processed polypeptide contains the pelB signal peptide, 734V_{H} and V_{K} coding sequences coupled by a 5 amino acid linker, and the carboxyl terminal 6 histidine affinity tag. The figure also shows a stick figure drawing of the mature polypeptide following proteolytic removal of the pelB leader peptide, and a stick figure drawing of a 734 heterodimer, including metal-chelate complex indium-DTPA binding sites.

FIG. 4 is the coding sequence of nucleic acids and encoded amino acids for 679-scFᵥ-L5. 1-66 is the coding sequence for the pelB leader peptide. 70-426 is the coding sequence for 679V_{H}. 427-441 is the coding sequence for the linker peptide (GGGGS) 442-780 is the coding sequence for 679V_{K}. 787-804 is the coding sequence for the 6 histidine affinity tag.

FIG. 5 is the coding sequence of nucleic acids and encoded amino acids for h679-scFᵥ-L5. 1-66 is the coding sequence for the pelB leader peptide. 70-426 is the coding sequence for h679V_{H}. 427-441 is the coding sequence for the linker peptide (GGGGS). 442-780 is the coding sequence for h679V_{K}. 787-804 is the coding sequence for the 6 histidine affinity tag.

FIG. 6 is the coding sequence of nucleic acids and encoded amino acids for hMN14-scFᵥ-L5. 1-66 is the coding sequence for the pelB leader peptide. 70-423 is the coding sequence for hMN14 V_{H}. 424-438 is the coding sequence for the linker peptide (GGGGS). 439-759 is the coding sequence for hMN14 V_{K}. 766-783 is the coding sequence for the 6 histidine affinity tag.

FIG. 7 is the coding sequence of nucleic acids and encoded amino acids for m734 V_{H} and V_{L}.

FIGS. 8A-8B is the DNA coding sequence and deduced amino acid sequence for the V_{H}-chain of TS1. 1-63 is the coding sequence for the pelB leader peptide. 90-405 is the coding sequence for hMN14 V_{H}. 469-819 is the coding sequence for m734 V_{H}. 866-1222 is the coding sequence for m679 V_{H}.

FIGS. 9A-9B is the DNA coding sequence and deduced amino acid sequence for the V_{L}-chain of TS1. 1-63 is the coding sequence for the pelB leader peptide. 70-408 is the coding sequence for m679 V_{K}. 452-768 is the coding sequence for m734 V_{L}. 829-1149 is the coding sequence for hMN14 V_{K}.

### DETAILED DESCRIPTION OF THE PREFERRED EMBODIMENTS

Unless otherwise specified, “a” or “an” means “one or more”.

One embodiment of this invention relates to multi-specific, multivalent binding proteins and methods of generating these agents from V_{H} and V_{L} domains. The binding protein has three or more binding sites where at least one binding site binds with a hapten moiety and at least two sites bind with target antigens. The present invention further relates to bispecific, trivalent heterodimers that have at least one binding site with affinity towards molecules containing a histamine-succinyl-glycyl (HSG) moiety and at least two binding sites with affinity towards carcinoembryonic antigen (CEA), and to trispecific, trivalent heterodimers that have at least one binding site with affinity towards molecules containing a HSG moiety, at least one binding sites with affinity towards CEA, and at least one binding site having affinity towards a metal-chelate complex indium-DTPA. Moreover, this invention relates to recombinant vectors useful for the expression of these functional heterodimers in a microbial host.

Structurally, whole antibodies are composed of one or more copies of an Y-shaped unit that contains four polypeptides chains. Two chains are identical copies of a polypeptide, referred to as the heavy chain, and two chains are identical copies of a polypeptide, referred to as the light chain. Each polypeptide is encoded by individual DNA or by connected DNA sequences. The two heavy chains are linked together by one or more disulfide bonds and each light chain is linked to one of the heavy chains by one disulfide bond. Each chain has a N-terminal variable domains, referred to as V_{H} and V_{L} for the heavy and the light chains, respectively, and the non-covalent association of a pair of V_{H} and V_{L}, referred to as the Fv fragment, forms one antigen-binding site.

Discrete Fv fragments are prone to dissociation at low protein concentrations and under physiological conditions [Glockshuber et al., Biochemistry (1990) 29: 1362-1367], and have limited use. To improve stability and enhance potential utility, recombinant single-chain Fv (scFv) fragments have been produced and studied extensively, in which the C-terminal of the V_{H} domain (or V_{L}) is joined to the N-terminal of the V_{L} domain (or V_{H}) via a peptide linker of variable length. [For a recent review, see Hudson and Kortt, J. Immunological methods (1999) 231: 177-189]. ScFv can be produced by methods disclosed in U.S. Pat. No. 4,946,778 (1990) and U.S. Pat. No. 5,132,405 (1992).

ScFvs with linkers greater than 12 amino acid residues in length (for example, 15-or 18-residue linkers) allow interacting between the V_{H} and V_{L} domains on the same chain and generally form a mixture of monomers, heterodimers and small amounts of higher mass multimers, [U.S. Pat. No. 4,642,334 (1987); Kortt et al., Eur. J. Biochem. (1994) 221: 151-157]. ScFvs with linkers of 5 or less amino acid residues, however, prohibit intramolecular pairing of the V_{H} and V_{L} domains on the same chain, forcing pairing with V_{H} and V_{L} domains on a different chain. Linkers between 3- and 12-residues form predominantly dimers [Atwell et al., Protein Engineering (1999) 12: 597-604]. With linkers between 0 and 2 residues, trimeric (termed triabodies), tetrameric (termed tetrabodies) or higher oligomeric structures of scFvs are useful; however, the exact patterns of oligomerization appear to depend on the composition as well as the orientation of the V-domains, in addition to the linker length. For example, scFvs of the anti-neuraminidase antibody NC 10 formed predominantly trimers (V_{H} to V_{L} orientation) or tetramers (V_{L} to V_{H} orientation) with 0-residue linkers [Dolezal et al., Protein Engineering (2000) 13: 565-574]. For scFvs constructed from NC10 with 1- and 2-residue linkers, the V_{H} to V_{L} orientation formed predominantly heterodimers [Atwell et al., Protein Engineering (1999) 12: 597-604]; in contrast, the V_{L} to V_{H} orientation formed a mixture of tetramers, trimers, dimers, and higher mass multimers [Dolezal et al., Protein Engineering (2000) 13: 565-574]. For scFvs constructed from the anti-CD19 antibody HD37 in the V_{H} to V_{L} orientation, the 0-residue linker formed exclusively trimers and the 1-residue linker formed exclusively tetramers [Le Gall et al., FEBS Letters (1999) 453: 164-168].

As the non-covalent association of two or more scFv molecules can form functional diabodies, triabodies and tetrabodies, which are multivalent but monospecific, a similar association of two or more different scFv molecules, if constructed properly, may form functional multispecific scFv multimers. Monospecific diabodies, triabodies, and tetrabodies with multiple valencies have been obtained using peptide linkers consisting of 5 amino acid residues or less. Bispecific diabodies are generally heterodimers of two different scFvs, each scFv comprises the V_{H} domain from one antibody connected by a short linker to the V_{L} domain of another antibody. Several bispecific diabodies have been made using a di-cistronic expression vector that contains in one cistron a recombinant gene construct comprising V_{H1}-linker-V_{L2} and in the other cistron a second recombinant gene construct comprising V_{H2}-linker-V_{L1}. [See Holliger et al., Proc. Natl. Acad. Sci. USA (1993) 90: 6444-6448; Atwell et al., Molecular Immunology (1996) 33: 1301-1302; Holliger et al., Nature Biotechnology (1997) 15: 632-631; Helfrich et al., Int. J. Cancer (1998) 76: 232-239; Kipriyanov et al., Int. J. Cancer (1998) 77: 763-772; Holiger et al., Cancer Research (1999) 59: 2909-2916]. Methods of constructing scFvs are disclosed in U.S. Pat. No. 4,946,778 (1990) and U.S. Pat. No. 5,132,405 (1992). Methods of producing multivalent, multispecific binding proteins based on scFv are disclosed in U.S. Pat. No. 5,837,242 (1998), U.S. Pat. No. 5,844,094 (1998) and WO-98/44001 (1998), for bispecific diabolides, and in PCT/DE99/01350 for tandem diabodies. Bispecific antibodies can be prepared by such methods as recombinant engineering, chemical conjugation, and quadroma technology. Methods of manufacturing scFv-based agents of multivalency and multispecificity by constructing two polypeptide chains, one comprising of the V_{H} domains from at least two antibodies and the other the corresponding V_{L} domains are disclosed in U.S. Pat. No. 5,989,830 (1999) and U.S. Pat. No. 6,239,259 (2001).

Alternative methods of manufacturing multispecific and multivalent antigen-binding proteins from V_{H} and V_{L} domains are disclosed in U.S. Pat. No. 5,989,830 and U.S. Pat. No. 6,239,259. Such multivalent and multispecific antigen-binding proteins are obtained by expressing a discistronic vector which encodes two polypeptide chains, with one polypeptide chain consisting of two or more V_{H} domains (from the same or different antibodies) connected in series by a peptide linker and the other polypeptide chain consisting of complementary V_{L} domains connected in series by a peptide linker.

More recently, a tetravalent tandem diabody (termed tandab) with dual specificity has also been reported [Cochlovius et al., Cancer Research (2000) 60: 4336-4341]. The bispecific tandab is a dimer of two identical polypeptides, each containing four variable domains of two different antibodies (V_{H1}, V_{L1}, V_{H2}, V_{L2}) linked in an orientation to facilitate the formation of two potential binding sites for each of the two different specificities upon self-association.

One embodiment of the present invention is a bispecific, trivalent targeting protein comprising two heterologous polypeptide chains associated non-covalently to form three binding sites, two of which have affinity for one target and a third which has affinity for a hapten that can be attached to a carrier for a diagnostic and/or therapeutic agent. In a preferred embodiment, the binding protein has two CEA binding sites and one HSG binding site. The bispecific, trivalent targeting agents have two different scFvs, one scFv contains two V_{H} domains from one antibody connected by a short linker to the V_{L} domain of another antibody and the second scFv contains two V_{L} domains from the first antibody connected by a short linker to the V_{H} domain of the other antibody. The methods for generating multivalent, multispecific agents from V_{H} and V_{L} domains provide that individual chains synthesized from a DNA plasmid in a host organism are composed entirely of V_{H} domains (the V_{H}-chain) or entirely of V_{L} domains (the V_{L}-chain) in such a way that any agent of multivalency and multispecificity can be produced by non-covalent association of one V_{H}-chain with one V_{L}-chain. For example, forming a trivalent, trispecific agent, the V_{H}-chain will consist of the amino acid sequences of three V_{H} domains, each from an antibody of different specificity, joined by peptide linkers of variable lengths, and the V_{L}-chain will consist of complementary V_{L} domains, joined by peptide linkers similar to those used for the V_{H}-chain. Since the V_{H} and V_{L} domains of antibodies associate in an anti-parallel fashion, the preferred method in this invention has the V_{L} domains in the V_{L}-chain arranged in the reverse order of the V_{H} domains in the V_{H}-chain, as shown in the diagram below.

V_{H}-chain: NH2-----V_{H}1-La-V_{H}2-Lb-V_{H}3----COOH

V_{L}-chain: NH2-----V_{L}3-Lb-V_{L}2-La-V_{L}1-----COOH

The peptide linkers La and Lb may be the same or different.

More variable domains can be included to increase the valency or the number of specificities. For example, the two polypeptides shown below can form a tetravalent bispecific dimer that is bivalent for each of the two specificities.

V_{H}-chain: NH2-----V_{H}1-La-V_{H}1-Lb-V_{H}2-Lc-V_{H}2----COOH

V_{L}-chain: NH2-----V_{L}2-Lc-V_{L}2-Lb-V_{L}1-La-V_{L}1-----COOH

The peptide linkers La, Lb, and Lc may be the same or different. It remains to be determined whether the order of the variable domains in each chain may be critical for retaining functional activity of each specificity.

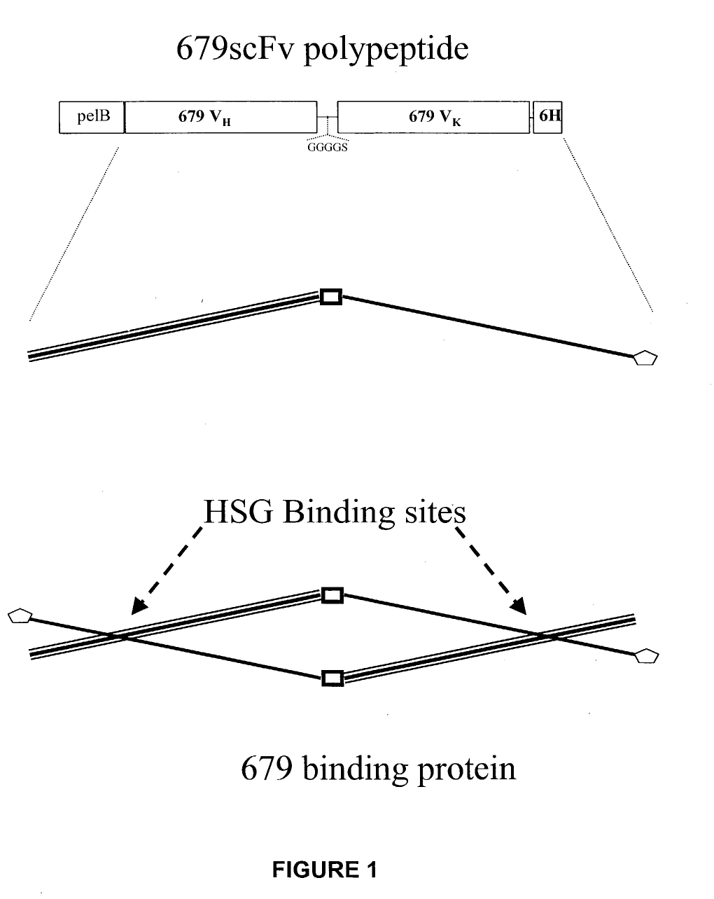

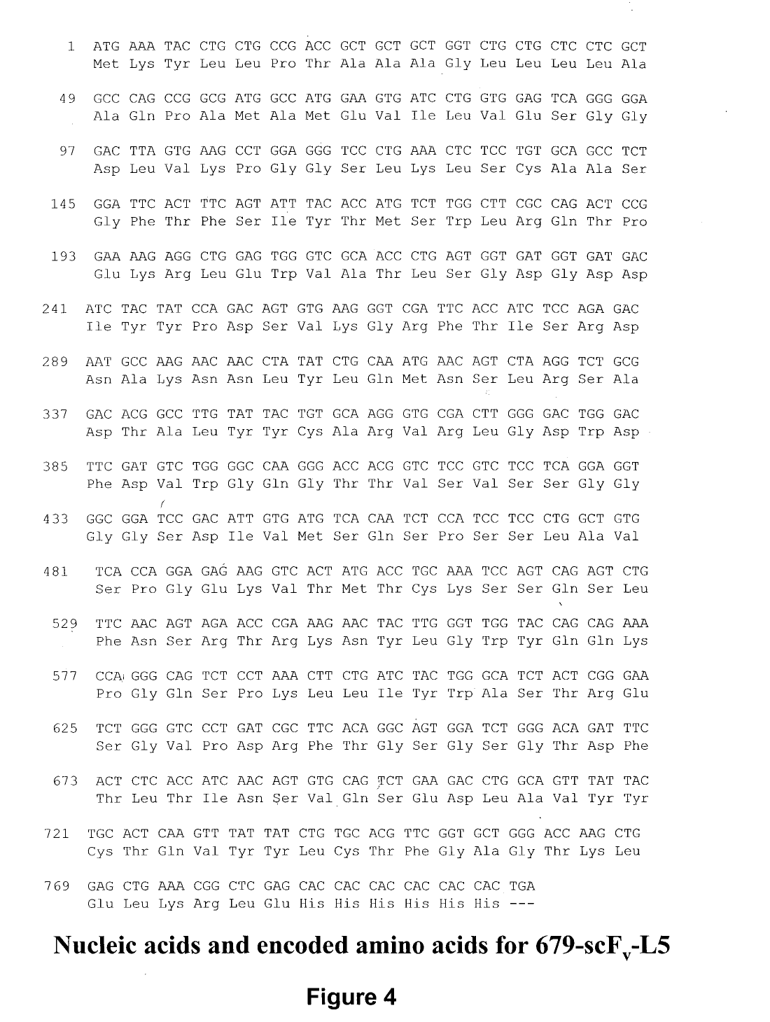

An additional embodiment of the present invention utilizes three monoclonal antibodies, 679, hMN14, and 734, to produce the V_{H} and V_{L} domains for constructing antigen specific heterodimers. Methods of making and using hMN14 and 734 are described in U.S. Ser. Nos. 09/337,756, 09/823,746 and 10/150,654, the contents of which are incorporated herein by reference in their entirety. The murine monoclonal antibody designated 679 (an IgG1, K) binds with high affinity to molecules containing the tri-peptide moiety histamine succinyl glycyl (HSG) (Morel et al, Molecular immunology, 27, 995-1000, 1990). The nucleotide sequence pertaining to the variable domains (V_{H} and V_{K}) of 679 has been determined (Qu et al, unpublished results). V_{K} is one of two isotypes of the antibody light chains, V_{L}. The function of the two isotypes is identical. As depicted in FIG. 1, the design of the gene construct (679-scFv-L5) for expressing a 679 heterodimer possesses the following features: 1) The carboxyl terminal end of V_{H} is linked to the amino terminal end of V_{K} by the peptide linker Gly-Gly-Gly-Gly-Ser (G₄S). The use of the G₄S peptide linker enables the secreted polypeptide to dimerize into a heterodimer, forming two binding sites for HSG. 2) A pelB leader signal peptide sequence precedes the V_{H} gene to facilitate the synthesis of the polypeptide in the periplasmic space of E. coli. 3) Six histidine (His) residues are added to the carboxyl terminus to allow purification by IMAC. The DNA coding sequence of nucleic acids and the corresponding encoded amino acids for 679-scFv-L5 are contained in FIG. 4 (Seq IDs). FIG. 1 also includes a stick figure drawing of the mature polypeptide after proteolytic removal of the pelB leader peptide and a stick figure drawing of a 679 heterodimer, including the HSG binding sites. 679 can be humanized or fully human to help avoid an adverse response to the murine antibody.

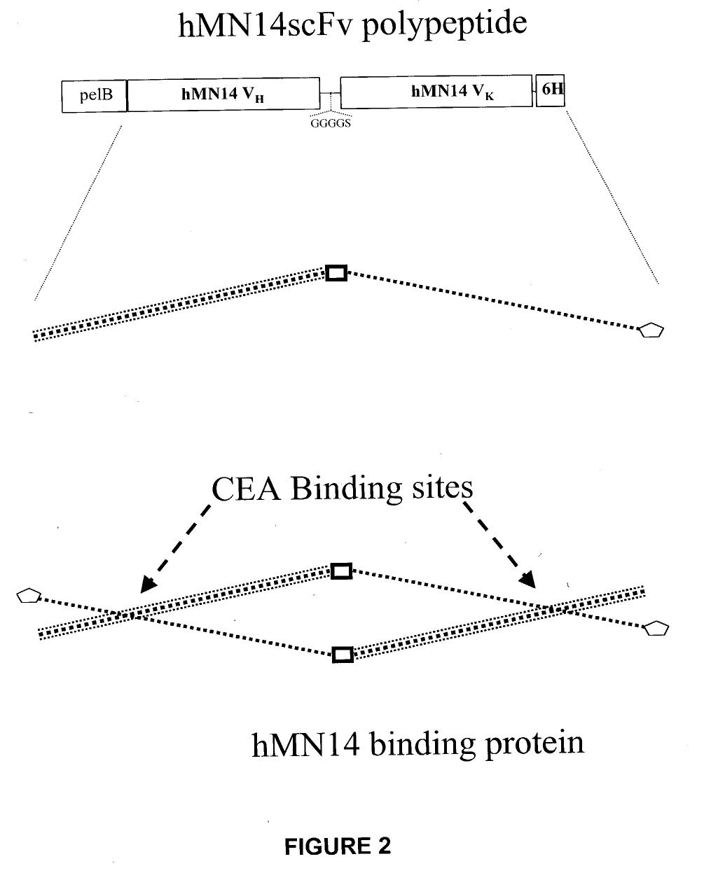

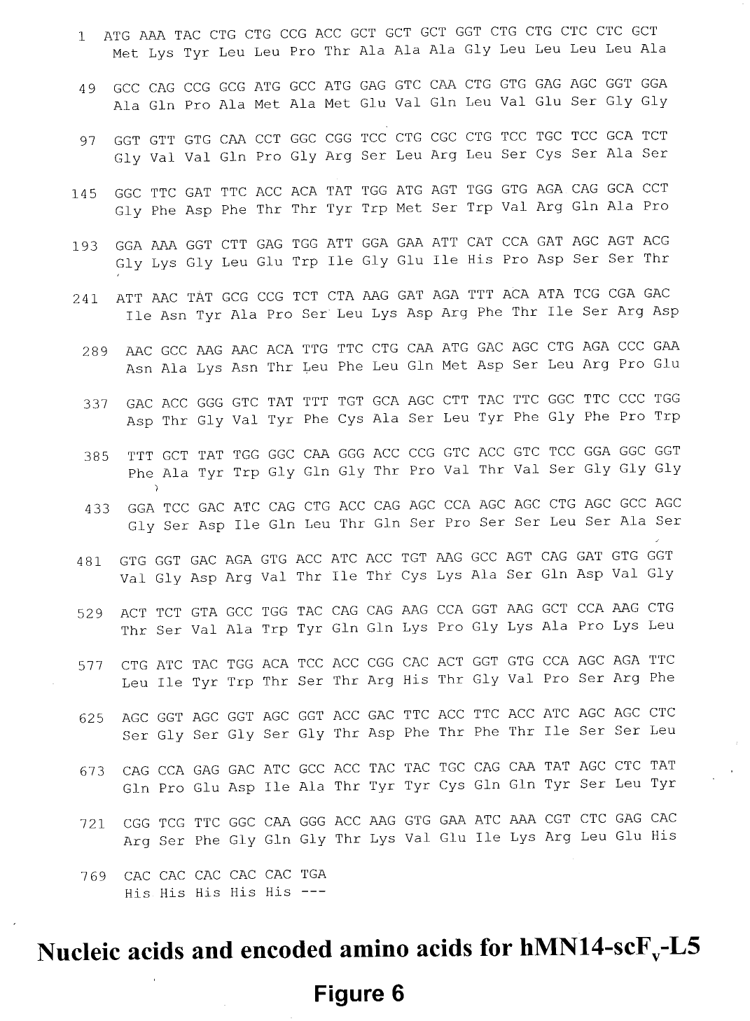

hMN14 is a humanized monoclonal antibody (Mab) that binds specifically to CEA (Shevitz et al, J. Nucl. Med., suppl., 34, 217P, 1993; U.S. Pat. No. 6,254,868 (2001)). While the original Mabs were murine, humanized antibody reagents are now utilized to reduce the human anti-mouse antibody response. The variable regions of this antibody were engineered into an expression construct (hMN14-scFv-L5). As depicted in FIG. 2, the design of the gene construct (hMN14-scFv-L5) for expressing an hMN14 heterodimer possesses the following features: 1) The carboxyl terminal end of V_{H} is linked to the amino terminal end of V_{K} by the peptide linker Gly-Gly-Gly-Gly-Ser (G₄S). The use of the G₄S peptide linker enables the secreted polypeptide to dimerize into a heterodimer, forming two binding sites for CEA. 2) A pelB leader sequence precedes the V_{H} gene to facilitate the synthesis of the polypeptide in the periplasmic space of E. coli. 3) Six histidine (His) residues are added to the carboxyl terminus to allow purification by IMAC. The DNA coding sequence of nucleic acids and the corresponding encoded amino acids for hMN14-scFv-L5 are contained in FIG. 6 (Seq IDs). FIG. 2 also shows a stick figure drawing of the mature polypeptide following proteolytic removal of the pelB leader peptide, and a stick figure drawing of a hMN14 heterodimer, including CEA binding sites.

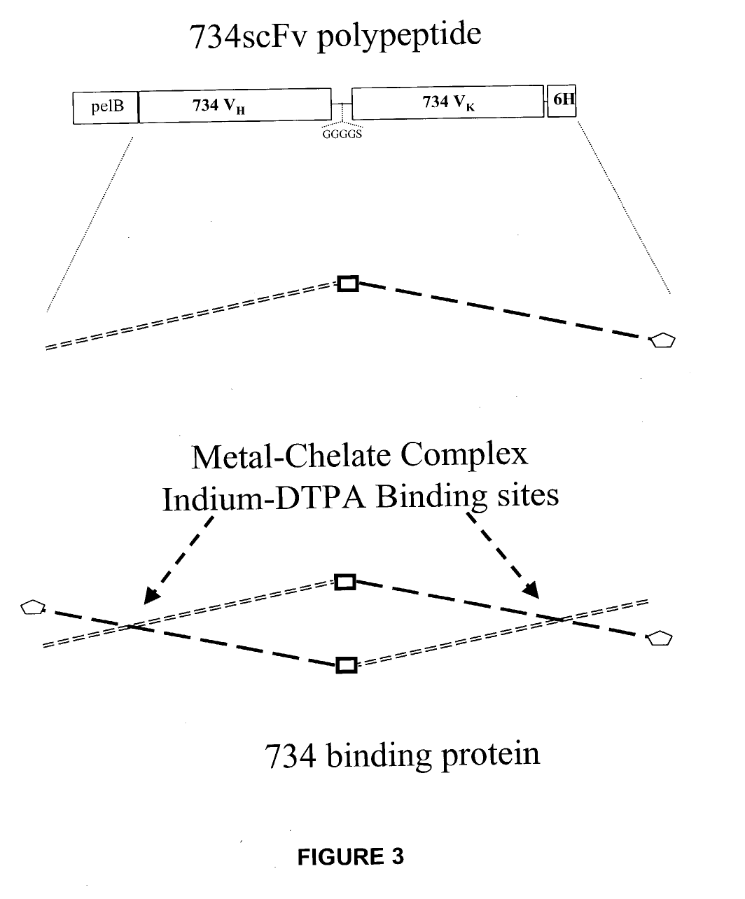

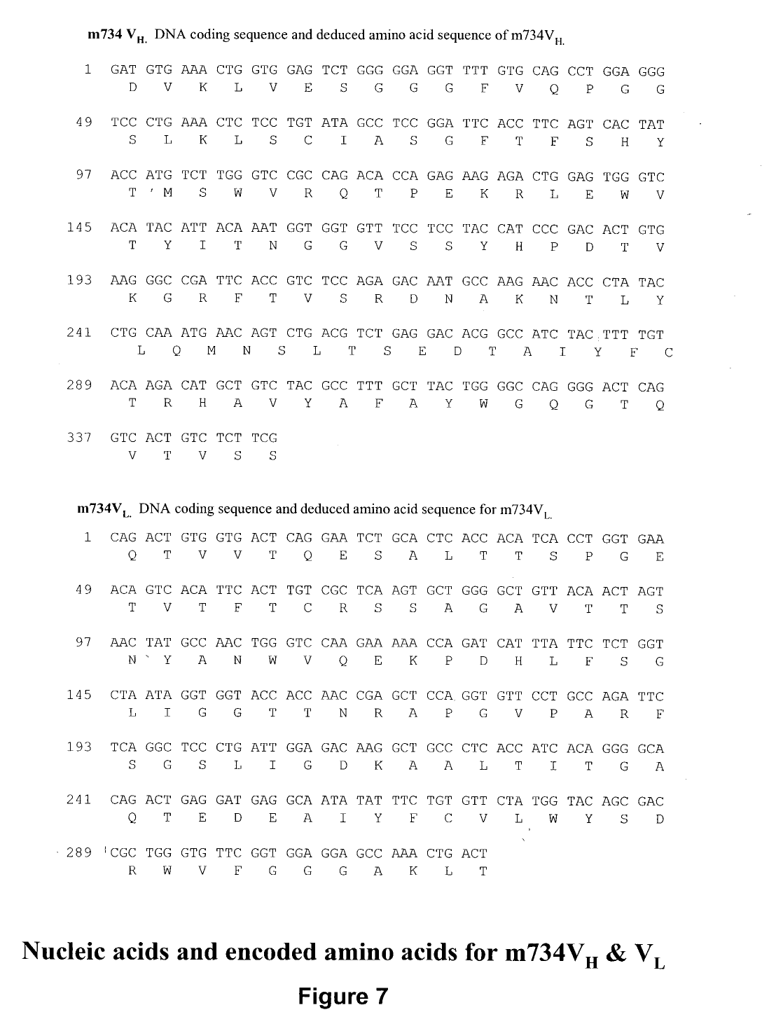

734 is a murine monoclonal antibody designated that binds with high affinity to the metal-chelate complex indium-DTPA (diethylenetriamine-pentaacetic acid). As depicted in FIG. 2, the design of the gene construct (734-scFv-L5) for expressing a 734 heterodimer possesses the following features: 1) The carboxyl terminal end of V_{H} is linked to the amino terminal end of V_{K} by the peptide linker Gly-Gly-Gly-Gly-Ser (G₄S). The use of the G₄S peptide linker enables the secreted polypeptide to dimerize into a heterodimer, forming two binding sites for HSG. 2) A pelB leader signal peptide sequence precedes the V_{H} gene to facilitate the synthesis of the polypeptide in the periplasmic space of E. coli. 3) Six histidine (His) residues are added to the carboxyl terminus to allow purification by IMAC. The DNA coding sequence of nucleic acids and the corresponding encoded amino acids for 734-scFv-L5 are contained in FIG. 7 (Seq IDs). FIG. 3 also includes a stick figure drawing of the mature polypeptide after proteolytic removal of the pelB leader peptide and a stick figure drawing of a 734 heterodimer, including the In-DTPA binding sites. 734 can be humanized or fully human to help avoid an adverse response to the murine antibody.

Di-cistronic expression vectors were constructed through a series of sub-cloning procedures. The di-cistronic expression cassette for trivalent bispecific 679xhMN14xhMN14 may be contained in a plasmid, which is a small, double-stranded DNA forming an extra-chromosomal self-replicating genetic element in a host cell. A cloning vector is a DNA molecule that can replicate on its own in a microbial host cell. This invention further includes a vector that expresses bispecific, trivalent heterodimers. A host cell accepts a vector for reproduction and the vector replicates each time the host cell divides. A commonly used host cell is Escherichia Coli (E. Coli), however, other host cells are available. The large production of recombinant antibody fragments available through host cell reproduction makes these antibodies a viable delivery system.

When the di-cistronic cassette is expressed in E. coi, some of the polypeptides fold and spontaneously form soluble bispecific, trivalent heterodimers. The bispecific, trivalent heterodimer shown has two polypeptides that interact with each other to form a HSG binding site having high affinity for HSG and four polypeptides that associate to form two CEA binding sites having high affinity for CEA antigens. Antigens are bound by specific antibodies to form antigen-antibody complexes, which are held together by the non-covalent interactions of the cross-linked antigen and antibody molecules. The trispecific, trivalent heterodimer has two polypeptides that interact with each other to form a HSG binding site having high affinity for HSG, two polypeptides that associate to form a CEA binding sites having high affinity for CEA antigens, and two polypeptides that associate to form a metal-chelate complex indium-DTPA binding site having high affinity for metal-chelate complex indium-DTPA.

Two constructs for expression of 679xhMN14xhMN14 bispecific heterodimers have been designed, constructed and tested. BS6 or BS8 (˜80 kDa) contain two binding sites for CEA and one binding site for HSG. BS6 differs from BS8 in the arrangement of respective V domains on the two polypeptides. The BS6 constituent polypeptides are hMN14V_{H}-(La)-hMN14V_{K}-(Lb)-679V_{H}-6His and 679V_{K}-(Lb)-hMN14V_{H}-(La)-hMN14V_{K}-6His. The polypeptides comprising BS8 are hMN14V_{H}-(L5)-hMN14V_{H}-(Lb)-679V_{H}-6His and 679V_{K}-(Lb)-hMN14V_{K}-(La)-hMN14V_{K}-6His. For BS6, the V_{H} polypeptide of the hMN14 MAb is connected to the V_{K} polypeptide of the hMN14 MAb by an oligopeptide linker, which is connected to the V_{H} polypeptide of the 679 MAb by an oligopeptide linker, and the V_{K} polypeptide of the 679 MAb is connected to the V_{H} polypeptide of the hMN14 MAb by an oligopeptide linker that is connected to the V_{K} polypeptide of the hMN14 MAB by an oligopeptide linker. Each chain forms one half of the 679xhMN14xhMN14 bispecific, trivalent heterodimer. BS8 is composed of the V_{H} polypeptide of the hMN14 MAb connected to the V_{H} polypeptide of the hMN14 MAb by an oligopeptide linker, which is connected to the V_{H} polypeptide of the 679 MAb by an oligopeptide linker and the V_{K} polypeptide of the 679 MAb connected to the V_{K} polypeptide of the hMN14 MAb by an oligopeptide linker, which is connected to the V_{K} polypeptide of the hMN14 MAb by an oligopeptide linker. Each chain forms one half of the 679xhMN14xhMN14 heterodimer. The oligopeptide linkers in BS6 and BS8 may be identical or different. The DNA coding sequence of nucleic acids and the corresponding encoded amino acids for the first and second polypeptide sequences of BS6 are hMN14V_{H}-(La)-hMN14V_{K}-(Lb)-679V_{H}-6His and 679V_{K}-(Lb)-hMN14V_{H}-(La)-hMN14V_{K}-6His, and for BS8 are hMN14V_{H}-(La)-hMN14V_{H}-(Lb)-679V_{H}-6His and 679V_{K}-(Lb)-hMN14V_{K}-(La)-hMN14V_{K}-6His, where hMN14 V_{H} and V_{K}, and 679 V_{H} and V_{K} are found in FIGS. 6 and 4 (SEQ ID), respectively.

The trispecific, trivalent binding protein, TS1, has one binding site for CEA, one binding site for HSG, and one binding site for metal-chelate indium-DTPA. The TS1 constituent polypeptides are hMN14V_{H}-(La)-734V_{H}-(Lb)-679V_{H} and 679V_{K}-(Lb)-734V_{K}-(La)-hMN14V_{K}. For TS1, the V_{H} polypeptide of the hMN14 MAb is connected to the V_{H} polypeptide of the 734 MAb by an oligopeptide linker, which is connected to the V_{H} polypeptide of the 679 MAb by an oligopeptide linker, and the V_{K} polypeptide of the 679 MAb is connected to the V_{K} polypeptide of the 734 MAb by an oligopeptide linker that is connected to the V_{K} polypeptide of the hMN14 MAB by an oligopeptide linker. Each chain forms one half of the hMN14x734x679 trispecific, trivalent heterodimer. The linkers may be identical or different. m734 V_{H} and V_{K} are found in FIG. 7 (SEQ ID).

The ultimate use of these bispecific, trivalent binding proteins is for pre-targeting CEA positive tumors for subsequent specific delivery of diagnostic or therapeutic agents carried by HSG containing peptides. These heterodimers bind selectively to two targeted antigens allowing for increased affinity and a longer residence time at the desired location. BS6 and BS8 are attractive pretargeting agents due to their ability to achieve higher levels of tumor uptake due to divalent CEA binding and longer circulation times. Moreover, non-antigen bound heterodimers are cleared from the body quickly and exposure of normal tissues is minimized. The diagnostic and therapeutic agents can include isotopes, drugs, toxins, cytokines, hormones, growth factors, conjugates, radionuclides, and metals. For example, gadolinium metal is used for magnet resonance imaging and MRI, CT, and ultrasound contrast agents are also utilized. Examples of radionuclides are, for example, ⁹⁰Y, ¹¹¹In, ¹³¹I, ^{99m}Tc, ¹⁸⁶Re, ¹⁸⁸Re, ¹⁷⁷Lu, ⁶⁷Cu, ²¹³Bi, ²¹³At. Other radionuclides are also available as diagnostic and therapeutic agents. Depending on the specificities engineered into these agents, potential applications are in cancer, autoimmune and infectious disease therapy, which may be achieved by invoking immune responses or in combination with AES technology using radioactive haptens or drug-hapten conjugates. Trispecific and tetraspecific agents may be useful in the detection and differentiation of specific target cells in blood samples.

Moreover, the present invention avoids the problem of forming multiple side-products because it only needs two complementary polypeptides to combine to form functional structures, and the identical polypeptides may never associate. Therefore, no inactive contaminants can form due to improper pairing of polypeptide chains. The present invention avoids the problem of intramolecular pairing because each polypeptide chain contains only V_{H} or V_{L} domains and therefore can form functional structures only when associated with the other polypeptide chain. The present invention avoids the problem of intramolecular pairing because each polypeptide chain contains only V_{H} or V_{L} domains (BS8 and TS 1), or they consist of an uneven number of V_{H} and V_{L} domains (BS6), and therefore can only form functional structures when associated with the complimentary chain. Although Davis et al. disclosed a similar approach (U.S. Pat. No. 5,989,830 (1999) and U.S. Pat. No. 6,239,259 (2001)) of constructing multivalent, multispecific proteins based on the pairing of two polypeptide chains, one comprising of the V_{H} domains from at least two antibodies and the other the corresponding V_{L} domains, little evidence establishing the molecular identity of each multivalent multispecific molecule was provided.

Delivering a diagnostic or a therapeutic agent to a target for diagnosis or treatment in accordance with the invention includes administering a patient with the binding protein, waiting a sufficient amount of time for an amount of the unbound protein to clear the patient's blood stream, and administering a diagnostic or therapeutic agent that binds to a binding site of the binding protein. Diagnosis further requires the step of detecting the bound proteins with known techniques. The diagnostic or therapeutic carrier molecule comprises a diagnostically or therapeutically efficient agent, a linking moiety, and one or more hapten moieties. The hapten moieties are positioned to permit simultaneous binding of the hapten moieties with the binding protein.

Administration of the binding protein and diagnostic or therapeutic agents of the present invention to a mammal may be intravenous, intraarterial, intraperitoneal, intramuscular, subcutaneous, intrapleural, intrathecal, by perfusion through a regional catheter, or by direct intralesional injection. When administering the binding moiety by injection, the administration may be by continuous infusion or by single or multiple boluses.

The unmixed diagnostic or therapeutic agent and bispecific antibody may be provided as a kit for human therapeutic and diagnostic use in a pharmaceutically acceptable injection vehicle, preferably phosphate-buffered saline (PBS) at physiological pH and concentration. The preparation preferably will be sterile, especially if it is intended for use in humans. Optional components of such kits include stabilizers, buffers, labeling reagents, radioisotopes, paramagnetic compounds, second antibody for enhanced clearance, and conventional syringes, columns, vials and the like.

The multivalent, multi-specific binding protein is useful for diagnosing and treating various human disorders, including cancer, autoimmune diseases, infectious diseases, cardiovascular diseases and inflammatory diseases. In this embodiment, the target antigen is a human disorder-associated binding site, such a cancer binding site, an autoimmune disease binding site, an infectious disease binding site, a cardiovascular disease binding site, and an inflammatory disease binding site.

Antibodies and antigens useful within the scope of the present invention include mAbs with properties as described above, and contemplate the use of, but are not limited to, in cancer, the following mAbs: LL1 (anti-CD74), LL2 (anti-CD22), RS7 (anti-epithelial glycoprotein-1 (EGP-1)), PAM-4 and KC4 (both anti-MUC1), MN-14 (anti-carcinoembryonic antigen (CEA)), Mu-9 (anti-colon-specific antigen-p), Immu 31 (an anti-alpha-fetoprotein), TAG-72 (e.g., CC49), Tn, J591 (anti-PSMA) and G250 (an anti-carbonic anhydrase IX mAb). Other useful antigens that may be targeted using these conjugates include HER-2/neu, BrE3, CD19, CD20 (e.g., C2B8, hA20, IF5 Mabs) CD21, CD23, CD80, alpha-fetoprotein (AFP), VEGF, EGF receptor, PlGF, MUC1, MUC2, MUC3, MUC4, PSMA, gangliosides, HCG, EGP-2 (e.g., 17-1A), CD37, HLD-DR, CD30, Ia, A3, A33, Ep-CAM, KS-1, Le(y), S100, PSA, tenascin, folate receptor, Thomas-Friedenreich antigens, tumor necrosis antigens, tumor angiogenesis antigens, Ga 733, IL-2, T101, MAGE, L243 or a combination thereof. A number of the aforementioned antibodies and antigens, as well as additional antibodies and antigens useful within the scope of the invention (e.g., anti-CSAP, MN-3 and anti-granulocyte antibodies), are disclosed in U.S. Provisional Application Serial No. 60/426,379, entitled “Use of Multi-specific, Non-covalent Complexes for Targeted Delivery of Therapeutics,” filed Nov. 15, 2002, U.S. Provisional Application Serial No. 60/360,229, entitled “RS7 Antibodies,” filed Mar. 1, 2002, U.S. Provisional Application Serial No. 60/356,132, entitled “Anti-CD20 Antibodies and Fusion Proteins Thereof and Methods of Use,” filed Feb. 14, 2002, U.S. Provisional Application Serial No. 60/333,479, entitled “Anti-DOTA Antibody,” filed Nov. 28, 2001, U.S. Provisional Application Serial No. 60/308,605, entitled “Polymeric Delivery Systems,” filed Jul. 31, 2001, U.S. Provisional Application Serial No. 60/361,037, entitled “Antibody point mutations for enhancing rate of clearance,” filed Mar. 1, 2002, U.S. Provisional Application Serial No. 60/360,259, entitled “Internalizing Anti-CD-74 Antibodies and Methods of Use,” filed Mar. 1, 2002, U.S. application Ser. No. 09/965,796, entitled “Immunotherapy of B-cell malignancies using anti-CD22 antibodies,” filed Oct. 1, 2001, U.S. Provisional Application Serial No. 60/60/342,104, entitled “Labeling Targeting Agents With Gallium-68 and Gallium-67,” filed Dec. 26, 2001, U.S. Application Serial No. 10/116,116, entitled “Labeling Targeting Agents With Gallium-68 and Gallium-67,” filed Apr. 5, 2002, U.S. Provisional Application Serial No. 60/399,707, entitled “Alpha-Fetoprotein Immu31 Antibodies and Fusion Proteins and Methods of Use Thereof,” filed Aug. 1, 2002, U.S. Provisional Application Serial No. 60/388,314, entitled “Monoclonal Antibody hPAM4,” filed Jun. 14, 2002, and U.S. Provisional Application Serial No. 60/414,341, entitled “Chimeric, Human and Humanized Anti-granulocyte Antibodies and Methods of Use,” filed Sep. 30, 2002, the contents of which are incorporated herein in their entirety.

In another preferred embodiment of the present invention, antibodies are used that internalize rapidly and are then re-expressed, processed and presented on cell surfaces, enabling continual uptake and accretion of circulating immunoconjugate by the cell. An example of a most-preferred antibody/antigen pair is LL1 an anti-CD74 mAb (invariant chain, class II-specific chaperone, Ii). The CD74 antigen is highly expressed on B-cell lymphomas, certain T-cell lymphomas, melanomas and certain other cancers (Ong et al., Immunology 98:296-302 (1999)), as well as certain autoimmune diseases.

The diseases that are preferably treated with anti-CD74 antibodies include, but are not limited to, non-Hodgkin's lymphoma, melanoma and multiple myeloma. Continual expression of the CD74 antigen for short periods of time on the surface of target cells, followed by internalization of the antigen, and re-expression of the antigen, enables the targeting LL1 antibody to be internalized along with any chemotherapeutic moiety it carries as a “payload.” This allows a high, and therapeutic, concentration of LL1-chemotherapeutic drug immunoconjugate to be accumulated inside such cells. Internalized LL1-chemotherapeutic drug immunoconjugates are cycled through lysosomes and endosomes, and the chemotherapeutic moiety is released in an active form within the target cells.

In another aspect, the invention relates to a method of treating a subject, comprising administering a therapeutically effective amount of a therapeutic conjugate of the preferred embodiments of the present invention to a subject. Diseases that may be treated with the therapeutic conjugates of the preferred embodiments of the present invention include, but are not limited to B-cell malignancies (e.g., non-Hodgkins lymphoma and chronic lymphocytic leukemia using, for example LL2 mAb; see U.S. Pat. No. 6,183,744), adenocarcinomas of endodermally-derived digestive system epithelia, cancers such as breast cancer and non-small cell lung cancer, and other carcinomas, sarcomas, glial tumors, myeloid leukemias, etc. In particular, antibodies against an antigen, e.g., an oncofetal antigen, produced by or associated with a malignant solid tumor or hematopoietic neoplasm, e.g., a gastrointestinal, lung, breast, prostate, ovarian, testicular, brain or lymphatic tumor, a sarcoma or a melanoma, are advantageously used.

### EXAMPLES

The examples below are illustrative of embodiments of the current invention and should not be used, in any way, to limit the scope of the claims.

### Example 1

Construction of Plasmids for Expression of BS8 in E. coli

Using the concept introduced in the present invention, a bispecific trivalent molecule (BS8) that is bivalent for CEA and monovalent for HSG was obtained by dimerization of the following two polypeptides:

V_{H}-chain: hMN14V_{H}-GGGGSGGGGSGGGGSM-hMN14V_{H}-GGGGS-679V_{H}

V_{L}-chain: 679V_{K}-GGGGS-hMN14V_{K}-LEGGGGSGGGGSGGGS-hMN14V_{K}

The DNA sequences for the two polypeptides were engineered into pET-ER vector, a di-cistronic bacterial expression plasmid, using standard molecular biology techniques. Upon expression, each polypeptide possesses an amino terminal pelB leader sequence that directs synthesis to the periplasmic space of E. coli and a carboxyl terminal six His affinity tag for purification by IMAC. We have demonstrated by BIAcore with a HSG coupled sensorchip by measuring the additional increase in response units upon successive injections of the bispecific agent followed by CEA or W12 (a rat anti-id monoclonal antibody to hMN14) that the two polypeptides indeed form a bispecific heterodimer that binds CEA divalently and HSG monovalently.

In this embodiment, the V polypeptide of the hMN14 MAb is connected to the V_{K} polypeptide of the hMN14 MAb by a five amino acid residue linker, which is connected to the V_{H} polypeptide of the 679 MAb by a sixteen amino acid residue linker, and the V_{K} polypeptide of the 679 MAb is connected to the V_{H} polypeptide of the hMN14 MAb by a sixteen amino acid residue linker that is connected to the V_{K} polypeptide of the hMN14 MAB by a five amino acid residue linker. Each chain forms one half of the 679xhMN14xhMN14 bispecific, trivalent heterodimer.

Alternatively, individual chains composed of both V_{H} and V_{L} domains can also be made to form multivalent, multispecific binding sites when paired. Such an example is provided by BS6 as described below.

### Example 2

Construction of Plasmids for Expression of BS6 in E. coli

Using a modification of the concept introduced in the present invention, an additional bi-specific trivalent molecule (BS6) that is bivalent for CEA and monovalent for HSG was obtained by dimerization of the following two polypeptides:

hMN14V_{H}-GGGGS-hMN14V_{K}-LEGGGGSGGGGSGGGS-679V_{H}

679V_{K}-GGGGSGGGGSGGGGSM-hMN14V_{H}-GGGGS-hMN14V_{K}

BS6 differs from BS8 in the arrangement of the domains in the specific polypeptide chains. Each chain of BS8 consists entirely of either V_{H} or V_{L} domains. The polypeptide chains of BS6 instead consist of two V_{H} and one V_{L} or one V_{H} and two V_{L}. In BS6, the linker between the hMN14V_{H} and hMN14V_{K} is only 5 amino acid residues in order to prevent their intra-chain association.

The DNA sequences for the two polypeptides of BS6 were engineered into pET-ER vector using standard molecular biology techniques. Upon expression, each polypeptide possesses an amino terminal pelB leader sequence and a carboxyl terminal six His affinity tag. We have demonstrated by BIAcore that the two polypeptides indeed form a bispecific heterodimer that binds CEA divalently and HSG monovalently.

In this embodiment, BS6 is composed of the V_{H} polypeptide of the hMN14 MAb connected to the V_{K} polypeptide of the hMN14 MAb by a five amino acid residue linker, which is connected to the V_{H} polypeptide of the 679 MAb by a sixteen amino acid residue linker and the V_{K} polypeptide of the 679 MAb connected to the V_{H} polypeptide of the hMN14 MAb by a sixteen amino acid residue linker, which is connected to the V_{K} polypeptide of the hMN14 MAb by a five amino acid residue linker. Each chain forms one half of the 679xhMN14xhMN14 bispecific, trivalent heterodimer.

### Example 3

Construction of Plasmids for Expression of TS1 in E. coli

Using the concept introduced in the present invention, a trispecific trivalent molecule (TS 1) that has binding moieties for CEA, HSG and In-DTPA was obtained by dimerization of the following two polypeptides:

V_{H}-chain: hMN14V_{H}-(L15)-734V_{H}-(L15)-679V_{H}

V_{L}-chain: 679V_{K}-(L15)-734V_{K}-(L15)-hMN14V_{K}

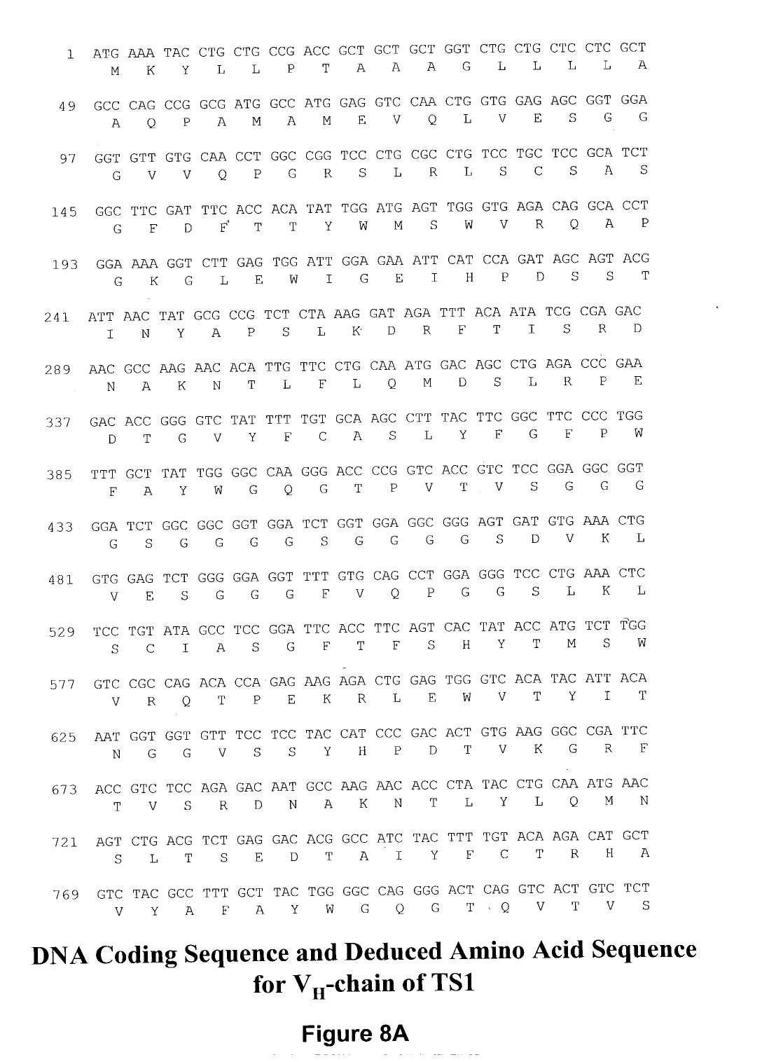

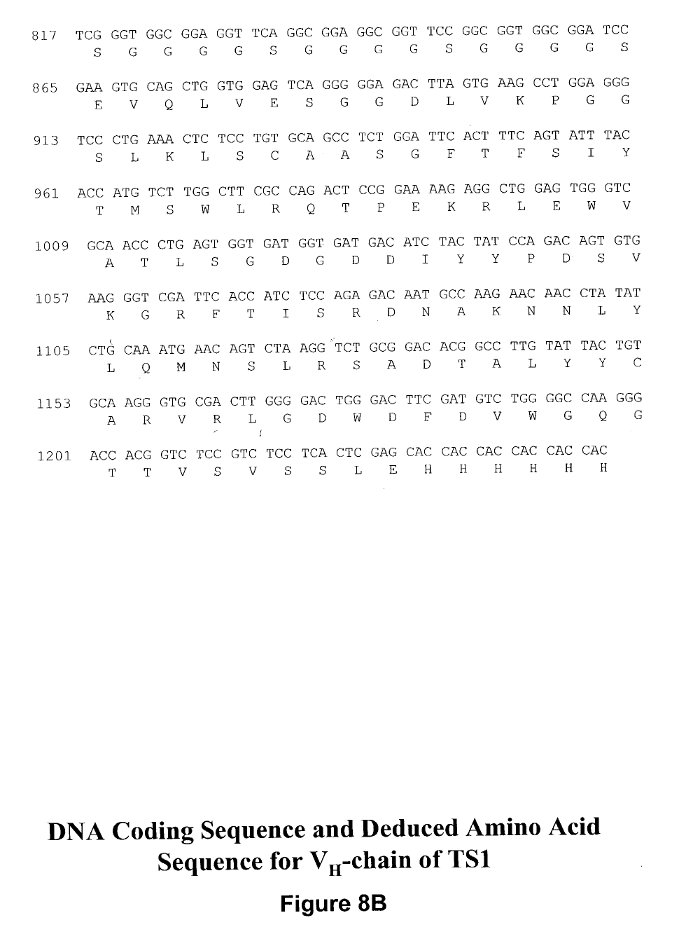

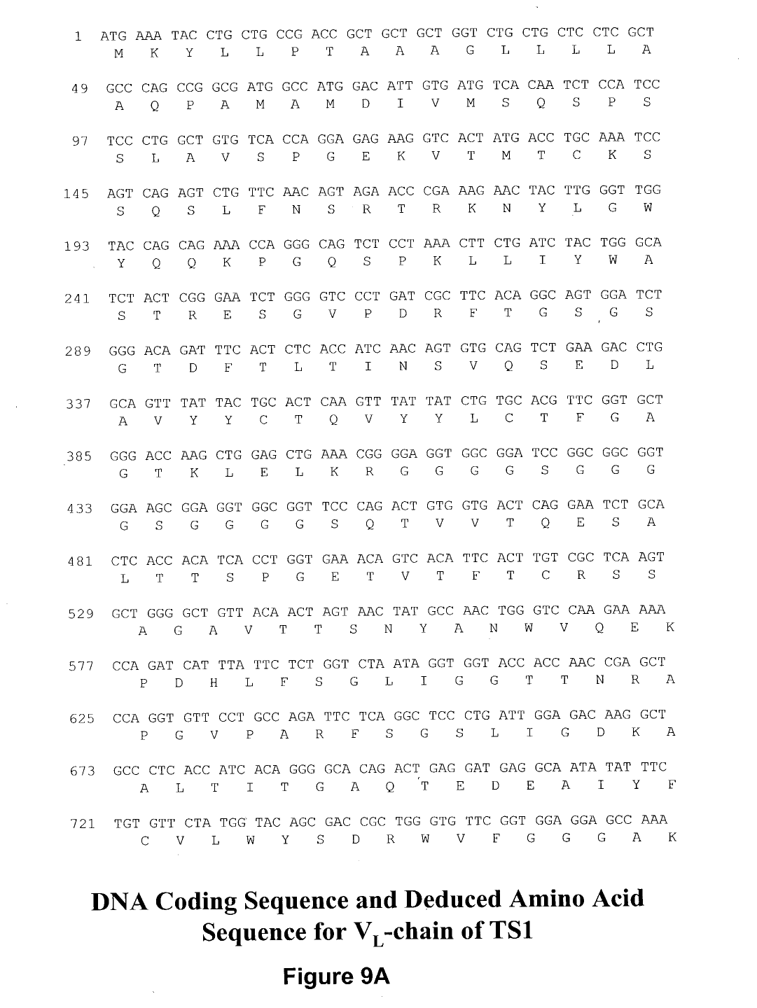

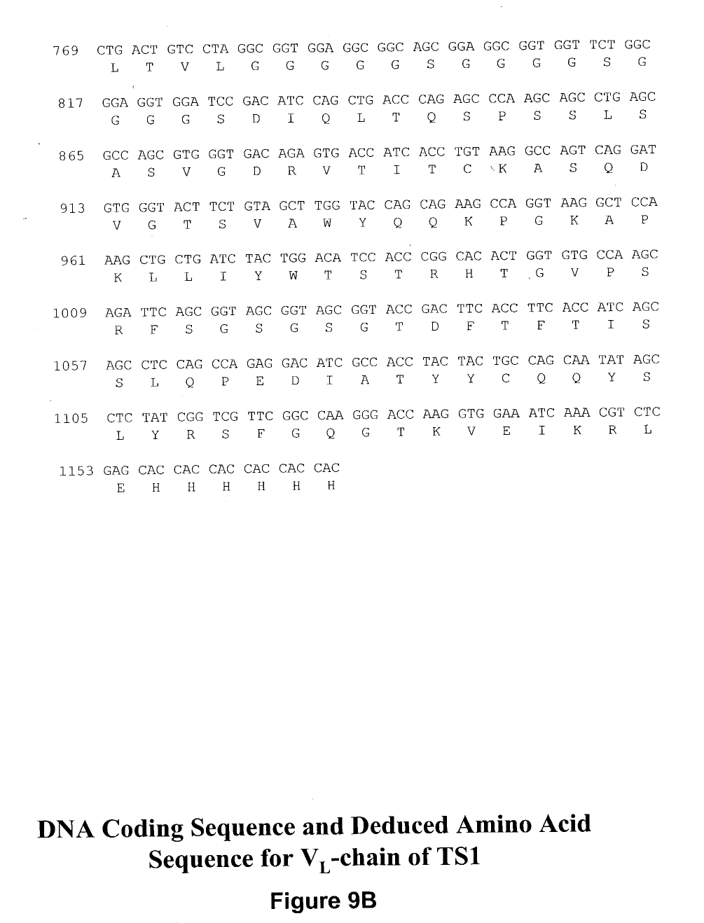

The DNA sequences for the two polypeptides were engineered into pET-ER vector using standard molecular biology techniques. (See FIGS. 8 and 9.) Upon expression, each polypeptide possesses an amino terminal pelB leader sequence that directs synthesis to the periplasmic space of E. coli and a carboxyl terminal six His affinity tag for purification by IMAC. We have demonstrated by BIAcore and ELISA that the two polypeptides indeed form a bispecific heterodimer with binding capabilities for CEA, HSG and In-DTPA.

In this embodiment, the V_{H} polypeptide of the hMN14 MAb is connected to the V_{H} polypeptide of the 734 MAb by a fifteen amino acid residue linker, which is connected to the V_{H} polypeptide of the 679 MAb by a fifteen amino acid residue linker, and the V_{K} polypeptide of the 679 MAb is connected to the V_{K} polypeptide of the hMN14 MAb by a fifteen amino acid residue linker that is connected to the V_{K} polypeptide of the hMN14 MAB by a fifteen amino acid residue linker. For TS 1, each 15 amino acid residue linker has the sequence Gly-Gly-Gly-Gly-Ser-Gly-Glyl-Gly-Gly-Ser-Gly-Glyl-Gly-Gly-Ser. Each chain forms one half of the hMN14x734x679 trispecific, trivalent heterodimer.

### Example 4

Uses of Multispecific, Multivalent Agents

The present invention is best used for the generation of in vivo targeting agents that can be trivalent bispecific, trivalent trispecific, tetravalent bispecific, tetravalent trispecific, or tetravalent tetraspecific. The trivalent bispecific (3-2S) agents will be derived from the variable domains of two different antibodies (V_{H1}/V_{L1} and V_{H2}/V_{L2}) and will be capable of binding to the antigens or epitopes recognized by the two antibodies. The binding will be bivalent for one specificity and monovalent for the other specificity. The 3-2S agents will be produced by dimerization of the two heterologous polypeptide chains shown in Diagram 1.

Diagram 1. Trivalent Bispecific Agents

V_{H}-chain: V_{H1}-La-V_{H1}-Lb-V_{H2}

V_{L}-chain: V_{L2}-Lc-V_{L1}-Ld-V_{L1}

The specific order of the three V_{H} or V_{L} domains may be varied and the peptide linkers (La, Lb, Lc, Ld) may be identical or different.

The trivalent trispecific (3-3S) agents will be derived from the variable domains of three different antibodies (V_{H1}/V_{L1}, V_{H2}/V_{L2}, and V_{H3}/V_{L3}) and will be capable of binding to the antigens or epitopes recognized by the three antibodies. The binding will be monovalent for each of the three different specificities. The 3-3S agents will be produced by dimerization of the two heterologous polypeptide chains shown in Diagram 2.

Diagram 2. Trivalent Trispecific Agents

V_{H}-chain: V_{H1}-La-V_{H2}-Lb-V_{H3}

V_{L}-chain: V_{L3}-Lc-V_{L2}-Ld-V_{L1}

The specific order of the three V_{H} or V_{L} domains may be varied and the peptide linkers (La, Lb, Lc, Ld) may be identical or different.

The tetravalent bispecific (4-2S) agents will be derived from the variable domains of two different antibodies (V_{H1}/V_{L1} and V_{H2}/V_{L2}) and will be capable of binding to the antigens or epitopes recognized by the two antibodies. The binding will be bivalent for each of the two different specificities. The 4-2S agents will be produced by dimerization of the two heterologous polypeptide chains shown in Diagram 3.

Diagram 3. Tetravalent Bispecific Agents

V_{H}-chain: V_{H1}-La-V_{H1}-Lb-V_{H2}-Lc-V_{H2}

V_{L}-chain: V_{L2}-Ld-V_{L2}-Le-V_{L1}-Lf-V_{L1}

The specific order of the four V_{H} or V_{L} domains may be varied and the peptide linkers (La, Lb, Lc, Ld, Le and Lf) may be identical or different.

The tetravalent trispecific (4-3S) agents will be derived from the variable domains of three different antibodies (V_{H1}/V_{L1}, V_{H2}/V_{L2}, and V_{H3}/V_{L3}) _{and} will be capable of binding to the antigens or epitopes recognized by the three antibodies. The binding will be bivalent for one of the three specificities and monovalent for each of the two other specificities. The 4-3S agents will be produced by dimerization of the two heterologous polypeptide chains shown in Diagram 4.

Diagram 4. Tetravalent Trispecific Agents

V_{H}-chain: V_{H1}-La-V_{H1}-Lb-V_{H2}-Lc-V_{H3}

V_{L}-chain: V_{L3}-Ld-V_{L2}-Le-V_{L1}-Lf-V_{L1}

The specific order of the four V_{H} or V_{L} domains may be varied and the peptide linkers (La, Lb, Lc, Ld, Le and Lf) may be identical or different.

The tetravalent tetraspecific (4-4S) agents will be derived from the variable domains of four different antibodies (V_{H1}/V_{L1}, V_{H2}/V_{L2}, V_{H3}/V_{L3}, and V_{H4}/V_{L4}) and will be capable of binding to the antigens or epitopes recognized by the four antibodies. The binding will be monovalent for each of the four specificities. The 4-4S agents will be produced by dimerization of the two heterologous polypeptide chains shown in Diagram 5.

Diagram 5. Tetravalent Tetraspecific Agents

V_{H}-chain: V_{H1}-La-V_{H2}-Lb-V_{H3}-Lc-V_{H4}

V_{L}-chain: V_{L4}-Ld-V_{L3}-Le-V_{L2}-Lf-V_{L1}

The specific order of the four V_{H} or V_{L} domains may be varied and the peptide linkers (La, Lb, Lc, Ld, Le and Lf) may be identical or different.

Antibodies of interest for producing these multivalent, multispecific agents include antibodies that exhibit high affinity for tumor associated antigens, such as CEA and MUC1, antibodies that exhibit high affinity for metal chelates, such as indium-DTPA, yttrium-DOTA, antibodies that exhibit high affinity for specific peptides, such as histamine-succinyl-glycine, antibodies that exhibit high affinity for cell differentiation antigens, such as CD20, CD22, CD74, antibodies that exhibit high affinity for enzymes, such as alkaline phosphatase, and antibodies that exhibit high affinity for cell surface markers of potential clinical utility, such as HLA-DR.

It will be apparent to those skilled in the art that various modifications and variations can be made to the compositions and processes of this invention. Thus, it is intended that the present invention cover such modifications and variations, provided they come within the scope of the appended claims and their equivalents.

The disclosure of all publications cited above are expressly incorporated herein by reference in their entireties to the same extent as if each were incorporated by reference individually.

What is claimed is:

1. A kit for delivering a diagnostic agent, a therapeutic agent, or a combination thereof to a target, comprising
a. a multivalent, multi-specific binding protein comprising three or more binding sites, wherein at least one binding site has affinity towards a hapten moiety and at least two binding sites have affinity towards a target antigen; and
b. a carrier molecule comprising a diagnostic agent, a therapeutic agent, or a combination thereof, a linking molecule, and at least two hapten moieties positioned to permit simultaneous binding of said hapten moieties with two of said binding proteins.

2. A multivalent, multi-specific binding protein comprising a first binding site having an affinity towards a hapten moiety and a second and a third binding site each having affinity towards a target antigen, which may the be the same or a different target antigen.

3. The binding protein of claim 2, wherein said target antigen is a human disorder-associated binding site.

4. The binding protein of claim 3, wherein said human disorder-associated binding site is selected from the group consisting of cancer binding sites, autoimmune disease binding sites, infectious disease binding sites, cardiovascular disease binding sites, and inflammatory disease binding sites.

5. The binding protein of claim 2, wherein said first binding site has an affinity towards molecules containing a histamine-succinyl-glycyl (HSG) moiety and said second and said third binding sites each have affinity towards carcinoembryonic antigen (CEA).

6. The binding protein of claim 5, wherein said binding protein comprises murine, humanized, or human sequences or a combination thereof.

7. The binding protein of claim 5, wherein said first binding site comprises a first and second polypeptide that associate with each other to form said HSG antigen binding site.

8. The binding protein of claim 7, wherein said first polypeptide comprises a V_{H} polypeptide of 679 MAb (FIG. 4, SEQ ID), and said second polypeptide comprises a V_{K} polypeptide of 679 MAb (FIG. 4, SEQ ID).

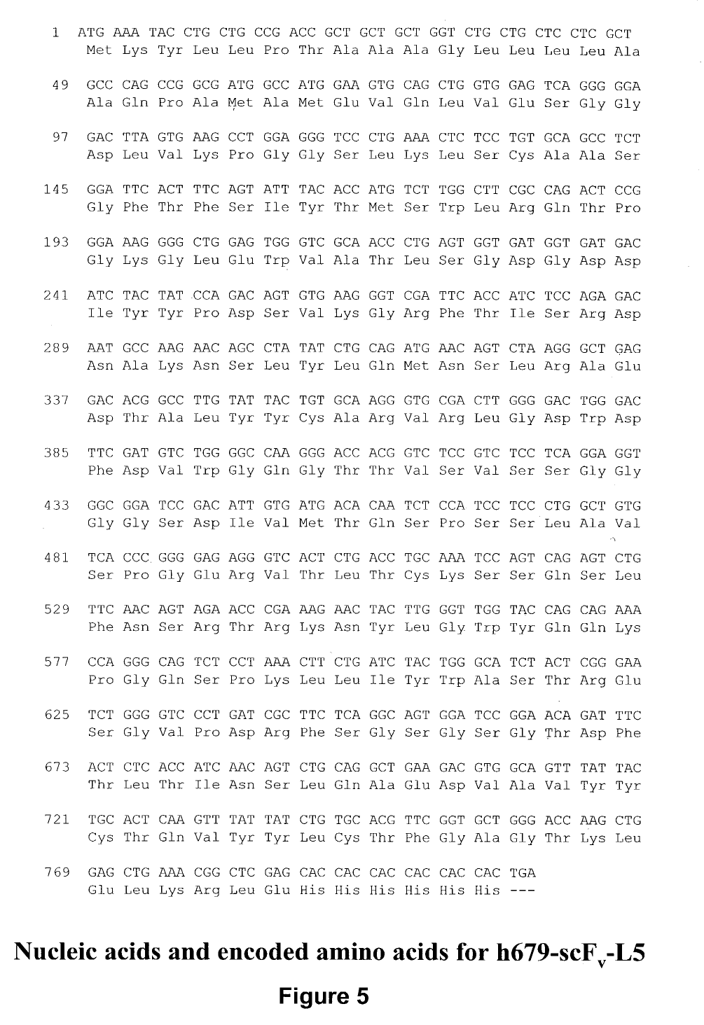

9. The binding protein of claim 7, wherein said first polypeptide comprises a V_{H} polypeptide of h679 MAb (FIG. 5, SEQ ID), and said second polypeptide comprises a V_{K} polypeptide of h679 MAb (FIG. 5, SEQ ID).

10. The binding protein of claim 8, wherein said second binding site comprises a third and fourth polypeptide that associate with each other to form said first CEA antigen binding site.

11. The binding protein of claim 10, wherein said third polypeptide comprises a V_{H} polypeptide of hMN14 MAb (FIG. 6, SEQ ID), and said fourth polypeptide comprises a V_{K} polypeptide of hMN14 MAb (FIG. 6, SEQ ID).

12. The binding protein of claim 8, wherein said third binding site comprises a fifth and sixth polypeptide that associate with each other to form said second CEA antigen binding site.

13. The binding protein of claim 12, wherein said fifth polypeptide comprises a V_{H} polypeptide of hMN14 MAb (FIG. 6, SEQ ID), and said sixth polypeptide comprises a V_{K} polypeptide of hMN14 MAb (FIG. 6, SEQ ID).

14. The binding protein of claim 12, wherein said first and fourth polypeptides are connected by a first linker, said fourth and fifth polypeptides are connected by a second linker, and said second and third polypeptides are connected by a third linker and said third and sixth polypeptides are connected by a fourth linker.

15. The binding protein of claim 14, wherein said first linker and said third linker each comprise sixteen amino acid residues and said second linker and said fourth linker each comprise five amino acid residues.

16. The binding protein of claim 12, wherein said first and third polypeptides are connected by a first linker, said third and fifth polypeptides are connected by a second linker, and said second and fourth polypeptides are connected by a third linker and said fourth and sixth polypeptides are connected by a fourth linker.

17. The binding protein of claim 16, wherein said first linker and said third linker each comprise sixteen amino acid residues and said second linker and said fourth linker each comprise five amino acid residues.

18. The binding protein of claim 5, wherein said binding protein is a heterodimer.

19. The binding protein of claim 12, wherein said first, second, third, fourth, fifth, and sixth polypeptides are each encoded by a first, second, third, fourth, fifth, and sixth cDNA, respectively.

20. The binding protein of claim 19, wherein said first, second, third, fourth, fifth, and sixth cDNA comprise nucleotide sequences shown in FIGS. 4 and 6 (SEQ ID).

21. A nucleic acid molecule comprising the first, fourth, and fifth cDNAs encoding the binding protein of claim 20.

22. A nucleic acid molecule comprising the second, third, and sixth cDNAs encoding the binding protein of claim 20.

23. An expression cassette comprising the nucleotide sequences encoding the binding protein of claim 20.

24. The expression cassette of claim 23, wherein said expression cassette is a plasmid.

25. A host cell comprising the plasmid of claim 24.

26. A method of producing a binding protein, comprising culturing the host cell of claim 25 in a suitable medium, and separating said binding protein from said medium.

27. The binding protein of claim 14, wherein said first, second, third, fourth, fifth, and sixth polypeptides are each encoded by a first, second, third, fourth, fifth, and sixth cDNA, respectively.

28. The binding protein of claim 27, wherein said first, second, third, fourth, fifth, and sixth cDNA comprise nucleotide sequences shown in FIGS. 4 and 6 (SEQ ID).

29. The binding protein of claim 27, wherein said first, third, and fifth cDNAs are on a first single nucleic acid molecule.

30. The binding protein of claim 29, wherein said second, fourth, and sixth cDNAs are on a second single nucleic acid molecule.

31. An expression cassette comprising the nucleic acid molecules encoding the binding protein of claim 30.

32. The expression cassette of claim 31, wherein said expression cassette is a plasmid.

33. A host cell comprising the plasmid of claim 32.

34. A method of producing a binding protein, comprising culturing the host cell of claim 33 in a suitable medium, and separating said binding protein from said media.

35. A carrier molecule, comprising a diagnostic agent, a therapeutic agent, or a combination thereof, a linking moiety, and two or more hapten moieties, wherein said hapten moieties are positioned to permit simultaneous binding of said hapten moieties with binding sites of one or more binding proteins.

36. A method of screening to determine the molar substitution ratio of hapten to carrier molecule, comprising purifying a mixture of carrier molecule following a hapten linkage reaction and exposing the purified mixture to a metal-binding assay to determine said molar substitution ratio.

37. A method of delivering a diagnostic agent, a therapeutic agent, or a combination thereof to a target, comprising:
a. administering to a subject in need thereof the binding protein of claim 5;
b. waiting a sufficient amount of time for an amount of the non-binding protein to clear the subject's blood stream; and
c. administering to said subject a carrier molecule comprising a diagnostic agent, a therapeutic agent, or a combination thereof, that binds to a binding site of the binding protein.

38. The method of claim 37, wherein said carrier molecule binds to one binding site of a first binding protein and to a second binding site of a second binding protein.

39. The method of claim 37, wherein said diagnostic agent or said therapeutic agent is selected from the group consisting of isotopes, drugs, toxins, cytokines, hormones, growth factors, conjugates, radionuclides, and metals.

40. The method of claim 39, wherein said isotopes are selected from the group consisting of ⁹⁰Y, ¹¹¹In, ¹³¹I, ^{99m}Tc, ¹⁸⁶Re, ¹⁸⁸Re, ¹⁷⁷Lu, ⁶⁷Cu, ²¹²Bi, ²¹³Bi, and ²¹¹At.

41. The method of claim 39, wherein said drugs are any pharmaceuticals that bind with a carrier molecule.

42. The method of claim 39, wherein said metals are selected from the group consisting of gadolinium and contrast agents.

43. The method of claim 42, wherein said contrast agents are selected from the group consisting of MRI contrast agents, CT contrast agents, and ultrasound contrast agents.

44. A method of detecting or treating a human disorder, comprising:
a. administering to a subject in need thereof with the binding protein of claim 2;
b. waiting a sufficient amount of time for an amount of unbound binding protein to clear the subject's blood stream; and
c. administering to said subject a carrier molecule comprising a diagnostic agent, a therapeutic agent, or a combination thereof, that binds to a binding site of the binding protein.

45. The method of claim 40, wherein said human disorder is selected from the group consisting of cancer, autoimmune diseases, infectious diseases, cardiovascular diseases, and inflammatory diseases.

46. A multivalent, multi-specific binding protein comprising a histamine-succinyl-glycyl (HSG) binding site having an affinity towards molecules containing a HSG moiety, a metal-chelate complex indium-DTPA binding site having an affinity towards metal-chelate complex indium-DTPA and a carcinoembryonic antigen (CEA) binding site each having affinity towards CEA.

47. The binding protein of claim 42, wherein said binding protein comprises murine, humanized, or human sequences.

48. The binding protein of claim 42, which is encoded by the nucleotide sequences of FIGS. 8 and 9.

49. A nucleic acid molecule comprising the nucleotide sequences of FIGS. 8 and 9.

50. An expression cassette comprising the nucleic acid molecule of claim 49.

51. The expression cassette of claim 50, wherein said expression cassette is a plasmid.

52. A host cell comprising the plasmid of claim 24.


SEQUENCE LISTING (19 sequences)

SEQ ID NO: 1 - DNA, 807 bp, Artificial Sequence
  CDS (1)..(804)
  Description of Artificial Sequence 679-scFv-L5 nucleotide sequence
atgaaatacctgctgccgaccgctgctgctggtctgctgctcctcgctgcccagccggcgatggccatggaagtgcagctggtggagtcagggggagacttagtgaagcctggagggtccctgaaactctcctgtgcagcctctggattcactttcagtatttacaccatgtcttggcttcgccagactccgggaaaggggctggagtgggtcgcaaccctgagtggtgatggtgatgacatctactatccagacagtgtgaagggtcgattcaccatctccagagacaatgccaagaacagcctatatctgcagatgaacagtctaagggctgaggacacggccttgtattactgtgcaagggtgcgacttggggactgggacttcgatgtctggggccaagggaccacggtctccgtctcctcaggaggtggcggatccgacattgtgatgacacaatctccatcctccctggctgtgtcacccggggagagggtcactctgacctgcaaatccagtcagagtctgttcaacagtagaacccgaaagaactacttgggttggtaccagcagaaaccagggcagtctcctaaacttctgatctactgggcatctactcgggaatctggggtccctgatcgcttctcaggcagtggatccggaacagatttcactctcaccatcaacagtctgcaggctgaagacgtggcagtttattactgcactcaagtttattatctgtgcacgttcggtgctgggaccaagctggagctgaaacggctcgagcaccaccaccaccaccactga

SEQ ID NO: 2 - protein, 268 aa, Artificial Sequence
  Description of Artificial Sequence 679-scFv-L5 protein sequence
MKYLLPTAAAGLLLLAAQPAMAMEVQLVESGGDLVKPGGSLKLSCAASGFTFSIYTMSWLRQTPGKGLEWVATLSGDGDDIYYPDSVKGRFTISRDNAKNSLYLQMNSLRAEDTALYYCARVRLGDWDFDVWGQGTTVSVSSGGGGSDIVMTQSPSSLAVSPGERVTLTCKSSQSLFNSRTRKNYLGWYQQKPGQSPKLLIYWASTRESGVPDRFSGSGSGTDFTLTINSLQAEDVAVYYCTQVYYLCTFGAGTKLELKRLEHHHHHH

SEQ ID NO: 3 - DNA, 807 bp, Artificial Sequence; identical to SEQ ID NO: 1
  CDS (1)..(804)
  Description of Artificial Sequence h679-scFv- L5 nucleotide sequence

SEQ ID NO: 4 - protein, 268 aa, Artificial Sequence; identical to SEQ ID NO: 2
  Description of Artificial Sequence h679-scFv- L5 protein sequence

SEQ ID NO: 5 - DNA, 786 bp, Artificial Sequence
  CDS (1)..(783)
  Description of Artificial Sequence hMN14-scFv- L5 nucleotide sequence
atgaaatacctgctgccgaccgctgctgctggtctgctgctcctcgctgcccagccggcgatggccatggaggtccaactggtggagagcggtggaggtgttgtgcaacctggccggtccctgcgcctgtcctgctccgcatctggcttcgatttcaccacatattggatgagttgggtgagacaggcacctggaaaaggtcttgagtggattggagaaattcatccagatagcagtacgattaactatgcgccgtctctaaaggatagatttacaatatcgcgagacaacgccaagaacacattgttcctgcaaatggacagcctgagacccgaagacaccggggtctatttttgtgcaagcctttacttcggcttcccctggtttgcttattggggccaagggaccccggtcaccgtctccggaggcggtggatccgacatccagctgacccagagcccaagcagcctgagcgccagcgtgggtgacagagtgaccatcacctgtaaggccagtcaggatgtgggtacttctgtagcctggtaccagcagaagccaggtaaggctccaaagctgctgatctactggacatccacccggcacactggtgtgccaagcagattcagcggtagcggtagcggtaccgacttcaccttcaccatcagcagcctccagccagaggacatcgccacctactactgccagcaatatagcctctatcggtcgttcggccaagggaccaaggtggaaatcaaacgtctcgagcaccaccaccaccaccactga

SEQ ID NO: 6 - protein, 261 aa, Artificial Sequence
  Description of Artificial Sequence hMN14-scFv- L5 protein sequence
MKYLLPTAAAGLLLLAAQPAMAMEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSGGGGSDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRLEHHHHHH

SEQ ID NO: 7 - DNA, 351 bp, Artificial Sequence
  CDS (1)..(351)
  Description of Artificial Sequence m734Vh nucleotide sequence
gatgtgaaactggtggagtctgggggaggttttgtgcagcctggagggtccctgaaactctcctgtatagcctccggattcaccttcagtcactataccatgtcttgggtccgccagacaccagagaagagactggagtgggtcacatacattacaaatggtggtgtttcctcctaccatcccgacactgtgaagggccgattcaccgtctccagagacaatgccaagaacaccctatacctgcaaatgaacagtctgacgtctgaggacacggccatctacttttgtacaagacatgctgtctacgcctttgcttactggggccaggggactcaggtcactgtctcttcg

SEQ ID NO: 8 - protein, 117 aa, Artificial Sequence
  Description of Artificial Sequence m734Vh protein sequence
DVKLVESGGGFVQPGGSLKLSCIASGFTFSHYTMSWVRQTPEKRLEWVTYITNGGVSSYHPDTVKGRFTVSRDNAKNTLYLQMNSLTSEDTAIYFCTRHAVYAFAYWGQGTQVTVSS

SEQ ID NO: 9 - DNA, 321 bp, Artificial Sequence
  CDS (1)..(321)
  Description of Artificial Sequence m734VL nucleotide sequence
cagactgtggtgactcaggaatctgcactcaccacatcacctggtgaaacagtcacattcacttgtcgctcaagtgctggggctgttacaactagtaactatgccaactgggtccaagaaaaaccagatcatttattctctggtctaataggtggtaccaccaaccgagctccaggtgttcctgccagattctcaggctccctgattggagacaaggctgccctcaccatcacaggggcacagactgaggatgaggcaatatatttctgtgttctatggtacagcgaccgctgggtgttcggtggaggagccaaactgact

SEQ ID NO: 10 - protein, 107 aa, Artificial Sequence
  Description of Artificial Sequence m734VL protein sequence
QTVVTQESALTTSPGETVTFTCRSSAGAVTTSNYANWVQEKPDHLFSGLIGGTTNRAPGVPARFSGSLIGDKAALTITGAQTEDEAIYFCVLWYSDRWVFGGGAKLT

SEQ ID NO: 11 - DNA, 1245 bp, Artificial Sequence
  CDS (1)..(1245)
  Description of Artificial Sequence nucleotide sequence for Vh-chain of TS1
atgaaatacctgctgccgaccgctgctgctggtctgctgctcctcgctgcccagccggcgatggccatggaggtccaactggtggagagcggtggaggtgttgtgcaacctggccggtccctgcgcctgtcctgctccgcatctggcttcgatttcaccacatattggatgagttgggtgagacaggcacctggaaaaggtcttgagtggattggagaaattcatccagatagcagtacgattaactatgcgccgtctctaaaggatagatttacaatatcgcgagacaacgccaagaacacattgttcctgcaaatggacagcctgagacccgaagacaccggggtctatttttgtgcaagcctttacttcggcttcccctggtttgcttattggggccaagggaccccggtcaccgtctccggaggcggtggatctggcggcggtggatctggtggaggcgggagtgatgtgaaactggtggagtctgggggaggttttgtgcagcctggagggtccctgaaactctcctgtatagcctccggattcaccttcagtcactataccatgtcttgggtccgccagacaccagagaagagactggagtgggtcacatacattacaaatggtggtgtttcctcctaccatcccgacactgtgaagggccgattcaccgtctccagagacaatgccaagaacaccctatacctgcaaatgaacagtctgacgtctgaggacacggccatctacttttgtacaagacatgctgtctacgcctttgcttactggggccaggggactcaggtcactgtctcttcgggtggcggaggttcaggcggaggcggttccggcggtggcggatccgaagtgcagctggtggagtcagggggagacttagtgaagcctggagggtccctgaaactctcctgtgcagcctctggattcactttcagtatttacaccatgtcttggcttcgccagactccggaaaagaggctggagtgggtcgcaaccctgagtggtgatggtgatgacatctactatccagacagtgtgaagggtcgattcaccatctccagagacaatgccaagaacaacctatatctgcaaatgaacagtctaaggtctgcggacacggccttgtattactgtgcaagggtgcgacttggggactgggacttcgatgtctggggccaagggaccacggtctccgtctcctcactcgagcaccaccaccaccaccac

SEQ ID NO: 12 - protein, 415 aa, Artificial Sequence
  Description of Artificial Sequence Protein sequence for Vh-chain of TS1
MKYLLPTAAAGLLLLAAQPAMAMEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSGGGGSGGGGSGGGGSDVKLVESGGGFVQPGGSLKLSCIASGFTFSHYTMSWVRQTPEKRLEWVTYITNGGVSSYHPDTVKGRFTVSRDNAKNTLYLQMNSLTSEDTAIYFCTRHAVYAFAYWGQGTQVTVSSGGGGSGGGGSGGGGSEVQLVESGGDLVKPGGSLKLSCAASGFTFSIYTMSWLRQTPEKRLEWVATLSGDGDDIYYPDSVKGRFTISRDNAKNNLYLQMNSLRSADTALYYCARVRLGDWDFDVWGQGTTVSVSSLEHHHHHH

SEQ ID NO: 13 - DNA, 1173 bp, Artificial Sequence
  CDS (1)..(1173)
  Description of Artificial Sequence Nucleotide sequence for VL-chain of TS1
atgaaatacctgctgccgaccgctgctgctggtctgctgctcctcgctgcccagccggcgatggccatggacattgtgatgtcacaatctccatcctccctggctgtgtcaccaggagagaaggtcactatgacctgcaaatccagtcagagtctgttcaacagtagaacccgaaagaactacttgggttggtaccagcagaaaccagggcagtctcctaaacttctgatctactgggcatctactcgggaatctggggtccctgatcgcttcacaggcagtggatctgggacagatttcactctcaccatcaacagtgtgcagtctgaagacctggcagtttattactgcactcaagtttattatctgtgcacgttcggtgctgggaccaagctggagctgaaacggggaggtggcggatccggcggcggtggaagcggaggtggcggttcccagactgtggtgactcaggaatctgcactcaccacatcacctggtgaaacagtcacattcacttgtcgctcaagtgctggggctgttacaactagtaactatgccaactgggtccaagaaaaaccagatcatttattctctggtctaataggtggtaccaccaaccgagctccaggtgttcctgccagattctcaggctccctgattggagacaaggctgccctcaccatcacaggggcacagactgaggatgaggcaatatatttctgtgttctatggtacagcgaccgctgggtgttcggtggaggagccaaactgactgtcctaggcggtggaggcggcagcggaggcggtggttctggcggaggtggatccgacatccagctgacccagagcccaagcagcctgagcgccagcgtgggtgacagagtgaccatcacctgtaaggccagtcaggatgtgggtacttctgtagcttggtaccagcagaagccaggtaaggctccaaagctgctgatctactggacatccacccggcacactggtgtgccaagcagattcagcggtagcggtagcggtaccgacttcaccttcaccatcagcagcctccagccagaggacatcgccacctactactgccagcaatatagcctctatcggtcgttcggccaagggaccaaggtggaaatcaaacgtctcgagcaccaccaccaccaccac

SEQ ID NO: 14 - protein, 391 aa, Artificial Sequence
  Description of Artificial Sequence Protein sequence for VL-chain of TS1
MKYLLPTAAAGLLLLAAQPAMAMDIVMSQSPSSLAVSPGEKVTMTCKSSQSLFNSRTRKNYLGWYQQKPGQSPKLLIYWASTRESGVPDRFTGSGSGTDFTLTINSVQSEDLAVYYCTQVYYLCTFGAGTKLELKRGGGGSGGGGSGGGGSQTVVTQESALTTSPGETVTFTCRSSAGAVTTSNYANWVQEKPDHLFSGLIGGTTNRAPGVPARFSGSLIGDKAALTITGAQTEDEAIYFCVLWYSDRWVFGGGAKLTVLGGGGGSGGGGSGGGGSDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRLEHHHHHH

SEQ ID NO: 15 - protein, 5 aa, Artificial Sequence
  Description of Artificial Sequence Synthetic linker peptide
GGGGS

SEQ ID NO: 16 - protein, 6 aa, Artificial Sequence
  Description of Artificial Sequence 6-His tag
HHHHHH

SEQ ID NO: 17 - protein, 16 aa, Artificial Sequence
  Description of Artificial Sequence Synthetic linker peptide
GGGGSGGGGSGGGGSM

SEQ ID NO: 18 - protein, 16 aa, Artificial Sequence
  Description of Artificial Sequence Synthetic linker peptide
LEGGGGSGGGGSGGGS

SEQ ID NO: 19 - protein, 15 aa, Artificial Sequence
  Description of Artificial Sequence Synthetic linker peptide
GGGGSGGGGSGGGGS